(19) United States
(12) Patent Application Publication
(10) Pub. No.: US 2013/0174858 A1
(43) Pub. Date: Jul. 11, 2013

(54) ANGIOGRAPHY SURGICAL DRAPE

(71) Applicant: MEDICAL CONCEPTS DEVELOPMENT, INC., Woodbury, MN (US)

(72) Inventor: Leland W. ANNETT, Tierra Verde, FL (US)

(73) Assignee: MEDICAL CONCEPTS DEVELOPMENT, INC., Woodbury, MN (US)

(21) Appl. No.: 13/659,779

(22) Filed: Oct. 24, 2012

Related U.S. Application Data
(63) Continuation of application No. 12/652,400, filed on Jan. 5, 2010, now abandoned.
(60) Provisional application No. 61/142,758, filed on Jan. 6, 2009.

(51) Int. Cl. A61B 19/08

(52) U.S. Cl. CPC A61B 19/088
USPC 128/853

(57) ABSTRACT

A surgical drape including a clear plastic substrate which is substantially impervious to liquids. The substrate is positionable over a patient with a first surface of the substrate generally engaging the patient. The substrate has an aperture formed therein which affords access to a location to which an incision is to be made in the patient. The drape further includes an absorbent lamina which overlies a second surface of the substrate which faces in a direction opposite that in which the first surfaces faces. A clear plastic patch, also substantially impervious to liquids, is sealed relative to the substrate along a line encircling the aperture. The patch can have a hole, either initially provided at manufacture or created by a surgeon during the procedure, which is at least partially coextensive with the aperture.

### TECHNICAL FIELD

The present invention deals broadly with the field of surgery and structures utilized to maintain a sterile field during the performance of surgical procedures. More narrowly, however, it deals with structures characterized as surgical drapes. The specific focus of the invention is an angiography drape typically used during the performance of procedures requiring the visualization of blood vessels following the injection of a radiopaque material.

### BACKGROUND OF THE INVENTION

Angiography surgical drapes are known in the prior art. One such prior art drape employs an elongated, typically rectangular, thin main sheet of plastic film 10. A thin, absorbent, non-woven material lamina 12, typically narrower in width than the main sheet, is adhered to an upwardly-facing surface 14 of the plastic film 10 opposite a surface 16 of the film 10 intended to engage a patient. In one embodiment, the non-woven material 12 is laminated to the plastic film 10 along a central axis of the film. The non-woven material 12 can extend the full length or some significant part of the length of the plastic film.

Preferably, the plastic film 10 is transparent and permits surgeons to easily view controls (not shown) of an angiography table that are mounted along one or more edges of the table when the drape is adhered to a patient lying on the table.

It is conventional to provide the laminate so formed with one or more holes 17. Such holes are formed in the laminate at locations conveniently distanced from ends of the laminate.

A clear film patch 18 is applied to the laminate over each hole formed therein. In order to maximize adherence, such a patch is made to be applied generally concentric with respect to the hole it overlies.

Such a clear film patch 18 is made from a thin piece of film which can be rectangular in shape and somewhat larger than the hole it overlies. In the case of such drapes known in the prior art, each clear film patch has one surface covered with pressure-sensitive adhesive. A patch 18 is adhesively attached, as practiced in the prior art, to the top surface 20 of the non-woven material 12. That is, the patch 18 is adhesively attached to the surface of the non-woven material 12 opposite the surface 22 which is laminated to the plastic film.

A clear film patch has a hole 24 formed therein, typically smaller than the hole in the laminate which it overlies. The application is typically concentric with the hole 17 in the laminate which is overlain. Consequently, when the clear film patch 18 is secured to the laminate, a zone of exposed adhesive surrounds the hole 17 in the laminate. This zone is formed on the underside of the drape.

The holes in the laminate may be round, oval or any other appropriate shape. Release liner paper (not shown) is placed over the exposed adhesive surface of the clear film patch 18. Such a release liner serves to protect the adhesive from being unwantedly stuck to drape surfaces when the drape is folded up and packaged for transportation and storage.

In applying the drape to a patient, the drape is unfolded while the patient typically lies face up on a surgery table. The axis of elongation of the drape is made to extend along the length of the table, and, typically, the drape runs the full length of the table with the non-woven material side of the laminate facing away from the patient. A hole or holes formed in the drape are disposed at appropriate surgery locations.

Typically in the performance of angiography procedures, at least one hole is located proximate the groin area of the patient. This enables placement of the hole and the affording of access to the femoral vein of the patient with a release liner in engagement with the patient. The release liner is, thereafter, removed to expose the adhesive area of the clear film patch. The adhesive area of the patch 18 is thereby able to be adhered to the patient's skin. With the drape so configured relative to the patient, a surgical procedure can be performed. Typically, this includes the making of an access wound in the patient's leg through the adhered opening 24 in the clear film patch 18. Such a procedure also typically includes the insertion of a catheter into the wound, the feeding of the catheter to an area adjacent the patient's heart, the injection of a contrast medium solution through the catheter into the heart vessels, and using a fluoroscope to ascertain the locations of vessel blockages which need to be repaired. After surgical work is completed, the catheter is withdrawn from the patient's artery. At that time, a substantial amount of blood and other fluids flow from the wound onto the top surface of the drape. They are there absorbed by the non-woven material 12 which comprises the laminar drape. As will be able to be seen in view of this discussion, the necessity for an absorbent non-woven material at the top surface of the drape is evident.

The construction of such a prior art device can, however, be problematic. The clear film patch 18, being adhesively attached to the top surface of the non-woven material, permits the fluids produced by the surgical procedure to flood the top surface of the clear patch 18. The fluids can, consequently, flow around the edges of the patch and seep through the non-woven material 12 to the underside of the drape as reflected by arrow 25. The patient and the table supporting the patient can, thereby, contaminate the top sterile surface of the drape by creating a path for microorganisms to invade sterile areas.

It is to these dictates and problems of the prior art that the present invention is directed. It is an angiography drape which, because of its construction, overcomes these problems.

### SUMMARY OF THE INVENTION

The present invention is a surgical drape which serves to inhibit the flowing of fluids generated by a surgical procedure, as discussed hereinbefore, around the peripheral edges of a patch which, in part, comprises the drape, and the seeping of those fluids through a non-woven material lamina to the underside of the drape. The drape includes a substrate which is substantially impervious to the passage of liquids therethrough. Such a substrate is positionable over a patient upon whom a surgical procedure is to be performed. A first surface of the substrate is generally in engagement with the patient when in use, and a second surface of the substrate faces generally upwardly in a direction opposite that in which the first surface faces. The substrate has an aperture formed therein, the aperture affording access to a location of intended incision during the surgical procedure. The drape further includes an absorbent lamina which overlies the second surface of the substrate. Further, the drape includes a patch, discussed cursorily above, which also is substantially impervious to liquids passing therethrough, which engages the substrate and is sealed relative thereto along a line encircling the aperture. The patch has a hole formed therein, the hole being at least partially coextensive with the aperture formed in the substrate.

In one embodiment of the invention, the drape is constructed such that an upwardly-facing surface of the patch engages a downwardly-facing surface of the substrate. In such a construction, the downwardly-facing surface of the patch engages and is adhered to the patient. An appropriate adhesive is applied to the downwardly-facing surface of the patch to effect adherence. The adhesive employed would be one of a number of adhesives known in the prior art as having an antimicrobial effect. It will be understood that the downwardly-facing surface of the patch could also, in another desirable embodiment, engage the upwardly-facing surface of the substrate.

If desired, the substrate can be made of a transparent or translucent material so that operating room bed controls can be viewed in order to facilitate operation thereof. Further, an angiography drape in accordance with the present invention can include a fibrous layer sprayed on the downwardly-facing surface of the substrate. Such a lamina can afford comfort to the patient upon whom the surgical procedure is being performed.

The present invention is thus an improved surgical drape which can be used in angiography applications. More specific features and advantages obtained in view of those features will become apparent with reference to the DETAILED DESCRIPTION OF THE INVENTION, appended claims, and accompanying drawing figures.

### BRIEF DESCRIPTION OF THE DRAWINGS

FIG. 1 is a perspective view of a portion of a drape known in the prior art;

FIG. 2 is an enlarged view of the structure circled in FIG. 1;

FIG. 3 is a view of the present invention similar to the FIG. 1 portrayal of the prior art; and

FIG. 4 is a view of the present invention similar to FIG. 2.

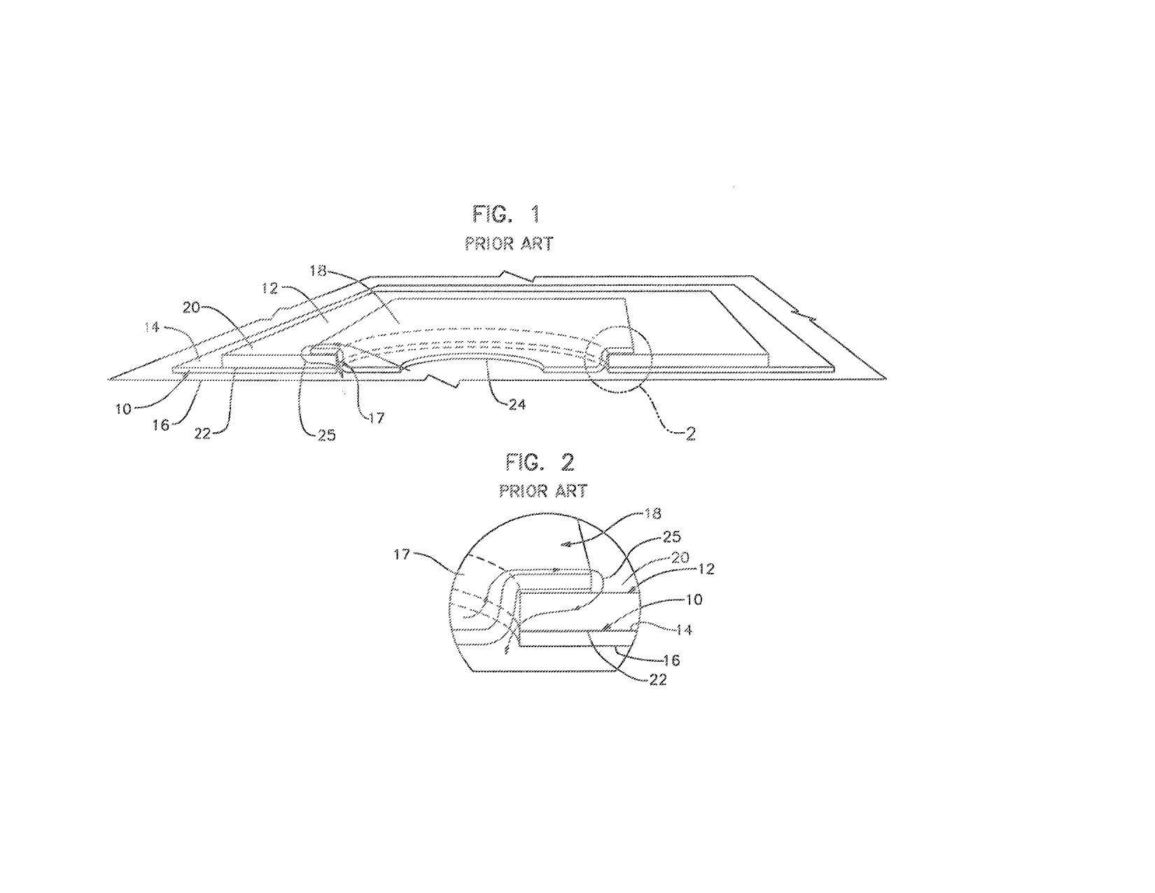

### DETAILED DESCRIPTION OF THE INVENTION

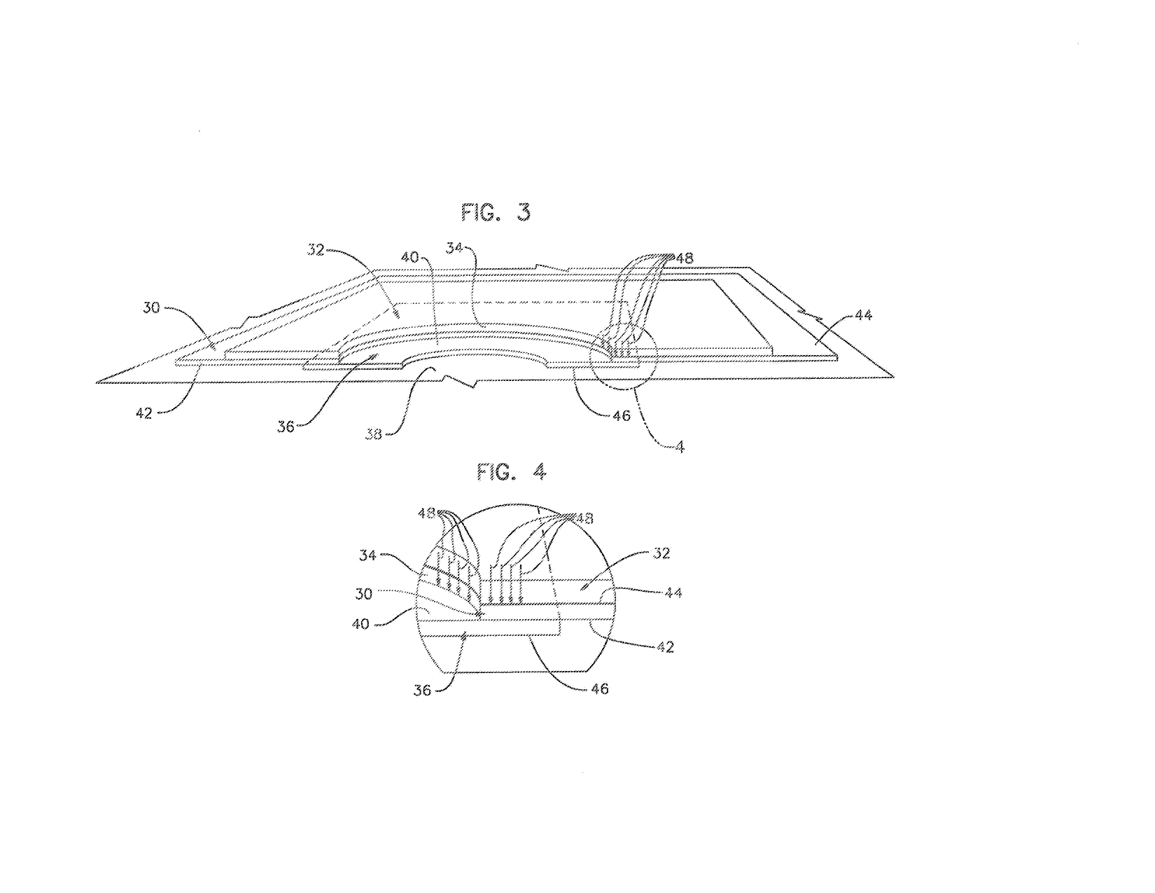

Referring now to the drawing figures wherein like reference numerals denote like elements throughout the several views, and focusing particularly on FIGS. 3 and 4, the improved surgical drape in accordance with the present invention is illustrated. As in the case of an angiography surgical drape known in the prior art, the present invention is intended to employ a sheet of plastic film 30. As in the prior art also, it is the intent that the plastic film be transparent or translucent in order to enable the surgeon to manipulate table/bed controls (not shown).

The present angiography drape also includes a layer of absorbent, non-woven material 32. The absorbent layer 32 is laminated to the plastic film 30 to form a laminate having a hole 34 defined therein.

A clear film patch 36 is shown as applied to the laminate with a hole 38 formed in the patch 36 being at least partially coextensive with the hole 34 formed in the plastic film 30/absorbent layer 32 laminate. FIG. 3 does, in fact, show circular holes 34, 38 which are concentric with respect to one another.

Unlike drapes known in the prior art, the present drape as shown in FIG. 3 has the patch 36 adhered to the plastic sheet 30. FIG. 3 shows the upper surface 40 of the patch 36 bonded to the lower surface 42 of the plastic sheet. Such bonding can be accomplished in any appropriate manner. It will be understood, however, that, since it is an objective of the present invention to maintain sterility where appropriate, bonding could be effected by means of an antimicrobial adhesive. The inventor of the present invention is also one of joint inventors in U.S. Pat. No. 6,503,531 issued on Jan. 7, 2003 for an ANTIMICROBIAL CONTAINING SOLVENTLESS HOT MELT ADHESIVE COMPOSITION. That patent describes usage of such an antimicrobial adhesive as might be employed in the present invention. The disclosure of U.S. Pat. No. 6,503,531 is hereby incorporated by reference.

Referring again to the structure of the present drape invention, an upper surface 44 of the plastic sheet 30 has the non-woven material lamina 32 applied thereto. Going from top to bottom, therefore, the layers of the drape include the non-woven lamina 32, the plastic sheet 30 and the patch 36 which is also substantially impervious to liquids passing therethrough. The bonding of the non-woven lamina 32 to the plastic sheet 30 occurs along a line which encircles the coextensive holes 34, 38. In the case of the present invention, an antimicrobial adhesive could, it is contemplated, be employed to bond the patch 36 to the lower side of the plastic sheet 42. The lower side 46 of the patch 36 would, during a surgical procedure, be secured, using an appropriate adhesive, to the patient. As a result of this structure being employed, fluids arising through the hole 38 in the patch 36 and the hole 34 in the plastic sheet/non-woven material lamina will be absorbed by the absorbent lamina 32. Their presence, however, will not contaminate any defined sterile area because of the seal between the patch 36 and the plastic lamina 30, and between the patch 36 and the skin of the patient. Penetration of the contaminated fluids, as illustrated by the arrows 48 in FIGS. 3 and 4, will be stopped by a dam comprised of the patch 36 and the plastic sheet 30. Those contaminated fluids will tend to be redirected back toward the non-woven absorbent material 32 to be held therein until subsequently disposed of.

It will be understood that this disclosure, in many respects, is only illustrative. Changes may be made in details, particularly in matters of shape, size, material, and arrangement of parts without exceeding the scope of the invention. Accordingly, the scope of the invention is as defined in the language of the appended claims.

1. A surgical drape, comprising:
a) a film substrate, substantially impervious to liquids, positionable over a patient upon whom a surgical procedure is to be performed with a first surface thereof generally engaging the patient, and a second surface thereof facing in a direction opposite that in which said first surface faces, said film substrate having an aperture, affording access to a location of intended incision, formed therein;
b) an absorbent material layer overlying said second surface of the film substrate; and
c) a patch, substantially impervious to liquids, engaging said film substrate and being sealed relative thereto along a line encircling said aperture, said patch having a hole, at least partially coextensive with said film substrate's aperture.

2. A surgical drape in accordance with claim 1 wherein said patch has first and second oppositely facing surfaces.

3. A surgical drape in accordance with claim 2 wherein said second surface of said patch engages said first surface of said film substrate and said first surface of said patch engages the patient.

4. A surgical drape in accordance with claim 3 wherein said first surface of said patch has adhesive applied thereto.

5. A surgical drape in accordance with claim 4 wherein said adhesive has antimicrobial properties.

6. A surgical drape in accordance with claim 5 wherein said adhesive comprises a matrix of an acrylic polymer in combination with an effective amount of diiodomethyl-p-tolylsulfone disposed throughout said adhesive.

7. A surgical drape in accordance with claim 2 wherein said first surface of said patch engages said second surface of said substrate.

8. A surgical drape in accordance with claim 1 wherein said substrate is made of a clear plastic.

9. A surgical drape in accordance with claim 8 wherein said substrate is flexible.

10. An angiography drape, comprising:
a1) a substrate, substantially impervious to liquids, positionable over a patient upon whom a surgical procedure is to be performed with a first surface thereof generally engaging the patient, and a second surface thereof facing in an opposite direction, said substrate having an aperture, affording access to a location of intended incision, formed therein;
b) a non-woven absorbent lamina overlying said second surface of said substrate; and
c) a patch, substantially impervious to liquids, having first and second opposing surfaces, with said second surface of said patch engaging said first surface of said substrate, said patch being sealed relative to said substrate along a line encircling said aperture with a hole formed in said patch being at least partially coextensive with said aperture.

11. An angiography drape in accordance with claim 10 further including a fibrous lamina sprayed on said first surface of said film substrate.

12. An angiography drape in accordance with claim 10 wherein said substrate and said patch are made of a clear plastic material.
